(19) United States
(12) Patent Application Publication
(10) Pub. No.: US 2020/0077958 A1
(43) Pub. Date: Mar. 12, 2020

(54) ELECTRICAL CONNECTION TO MINIATURE SENSORS

(71) Applicant: KONINKLIJKE PHILIPS N.V., EINDHOVEN (NL)

(72) Inventors: Johannes Wilhelmus WEEKAMP, Beek en donk (NL); Hendrik DRENTH, Veldhoven (NL)

(21) Appl. No.: 16/463,884

(22) PCT Filed: Nov. 22, 2017
(86) PCT No.: PCT/EP2017/080111

§ 371 Date: May 24, 2019

(30) Foreign Application Priority Data
Nov. 28, 2016 (EP) 16200930.2

(51) Int. Cl. A61B 5/00; A61B 5/01; A61B 8/12; H01B 7/02; H01B 7/08

(52) U.S. Cl. CPC A61B 5/6851; A61B 5/01; A61B 8/12; H01B 7/02; A61B 2562/222; A61B 2562/028; A61B 2562/0247; A61B 2562/227; H01B 7/0807

(57) ABSTRACT

A conductor arrangement is provided for connection to a miniature sensor. First and second conductor wires are spaced by a spacer. The maximum lateral dimension of the conductor arrangement, perpendicular to the wire length direction, is less than 500 μm. The spacing means that wire bending processes do not need to be carried out in order to make connection to the miniature sensor.

### FIELD OF THE INVENTION

This invention relates to miniature sensors, and in particular to the electrical connections to such sensors. One example is sensors mounted at the end of a guidewire.

### BACKGROUND OF THE INVENTION

Guidewires can be used for advancing catheters to a target region during a minimally invasive intervention. For example, guidewires are used to advance a catheter to a heart during a minimally invasive cardiovascular intervention.

Many minimally invasive cardiovascular interventions are performed with catheters, which are long thin tubes that can be advanced through the blood vessels with diagnostic or therapeutic payloads (e.g., contrast agents, pressure transducers, balloon, stents, etc.).

Due to a variety of reasons, for example to tolerate tortuosity of the vessel shape or vessel blockages, a guidewire may be advanced to a target region of the intervention prior to the introduction of the diagnostic or therapeutic catheter.

The guidewire is typically a thin wire with specifically designed material properties that facilitates a loading of the diagnostic or therapeutic catheter over an end of the guidewire and an advancement of the catheter over the guidewire to reach the target region.

These procedures are generally guided with real-time X-ray imaging, which depicts two-dimensional (“2D”) projection images of the catheters and guidewires. However, challenges with X-ray imaging include the 2D nature of the imaging and the ionizing radiation to the patient and physician.

A known alternative is optical shape sensing technology, which may provide full three-dimensional (“3D”) shape information of medical instruments without the need for any harmful radiation. It is known for example to implement spatially sensitive bend and twist sensing using optical fibers by combining multiple cores of fiber-bragg-grating (“FBG”) fibers in specific geometric orientations over distance.

It is also known to provide optical sensors as part of a guide wire and to pass signals along optical fibers which extend along the length of the guidewire. For example, fractional flow reserve (FFR) sensing may be implemented using fiber optic sensing. Thus, the use of the guidewire itself for sensing purposes is well known.

It is also known to provide electrical sensors at the tip of the guidewire, such as CMUT (capacitive micromachined ultrasound transducer) devices for measuring flow, pressure sensing piezo-crystals or temperature sensors. Sensors may also be used at the tip of a guidewire as a tracking device, for example by picking up ultrasound signals from an ultrasound probe. Based on the timing and direction of the ultrasound waves the position inside the body can be calculated. PVDF transducers may be used for this purpose.

This invention relates in particular to electrical sensors and the electrical connections that need to be made to such electrical sensors.

The sensor at the tip needs to be connected to signal carrying conductor lines, and these connections need to be made in a small space. By way of example the width of the set of conductor lines may be of the order of 100-200 micrometers. Furthermore, conductor line may provide power to the sensor.

The set of conductor lines may typically comprise a pair of side-by-side insulated electrical wires. These need to be stripped back at their ends and the bare wires are then conventionally spread apart and connected to pads of the sensor module by soldering or welding.

Due to the dimensions involved, this connection process is complicated and the required wire bending to connect to the sensor pads can decrease the wire quality and hence the product quality.

There is therefore a need for an improved electrical connection arrangement for miniature sensors of this type.

### SUMMARY OF THE INVENTION

The invention is defined by the claims.

According to examples in accordance with an aspect of the invention, there is provided a conductor arrangement for connection to a miniature sensor, comprising:

a first conductor wire;

a second conductor wire;

a dummy wire forming an insulating spacer between and bonded to the first and second conductor wires, wherein the first and second conductor wires and the dummy wire form a flat conductor array,

wherein the maximum lateral dimension of the conductor arrangement, perpendicular the wire length direction, is less than 500 μm.

This conductor arrangement provides a spaced pair of wires. The spacing means that wire bending processes do not need to be carried out in order to make connection to the miniature sensor. It is particularly desirable to avoid this wire bending process for miniature sensors, as a result of the small wire dimensions, and hence susceptibility to damage.

The first and second conductor wires preferably each comprise a metal core and an insulating shroud. Each metal core for example has a diameter of less than 100 μm, for example less than 50 μm and each insulating shroud has thickness of less than 40 μm, for example less than 20 μm.

The spacer may comprise an insulating cylinder. This insulating cylinder may be considered to be a dummy wire between the two conductor wires, and it has a diameter which is chosen to achieve the desired spacing between the conductor wires. The diameter of the dummy wire is preferably the same as for the conductor wires, so that the overall arrangement is then formed by bonding a set of dimensionally equivalent cylinders (some conducting and one or more not conducting) together. If more space is needed a second dummy wire can be added.

The use of a dummy wire provides a practical way to provide a uniform spacing. In particular, any angular orientation of the dummy wire can be permitted when combining the dummy wire with the conductor wires to form the overall structure. It simplifies the manufacture of the overall conductor arrangement at the desired small dimensions.

There may be exactly two conductor wires.

The invention also provides a sensor arrangement comprising:

a sensor module, having first and second connection pads; and

a conductor arrangement as defined above, wherein the first and second conductor wires each comprise a bare end portion which is connected to a respective one of the connection pads.

This defines a connected sensor module and conductor arrangement. The bare end portions of the conductor wires are preferably straight so that no bending is needed to make the connections to the sensor.

Each bare end portion may be connected to the respective connection pad by soldering or welding.

The pitch of the first and second conductor wires is preferably equal to a pitch of the first and second connection pads. This means that no bending is required. The pitch is for example between 30 and 200 μm.

The sensor module for example comprises a pressure sensor and/or a flow sensor and/or a temperature sensor.

The sensor arrangement may comprise a guidewire sensor arrangement. The sensor information is then used prior to a procedure making use of a catheter which is fed over the guidewire.

The invention also provides a guidewire for a catheter, comprising a sensor arrangement as defined above.

The invention also provides a catheter or stent system, comprising:

a guidewire as defined above; and

a catheter or stent which is adapted to be guided over the guidewire.

### BRIEF DESCRIPTION OF THE DRAWINGS

Examples of the invention will now be described in detail with reference to the accompanying drawings, in which:

FIG. 1 shows a conventional connection between a conductor arrangement and a sensor;

FIG. 2 shows a connection between a conductor arrangement in accordance with an example of the invention and a sensor to form a sensor arrangement;

FIG. 3 shows the conductor arrangement of FIG. 2 in cross section;

FIG. 4 shows the guidewire in cross section; and

FIG. 5 shows a guidewire and catheter system using the sensor arrangement of FIG. 2.

### DETAILED DESCRIPTION OF THE EMBODIMENTS

The invention provides a conductor arrangement for connection to a miniature sensor. First and second conductor wires are spaced by a spacer. The maximum lateral dimension of the conductor arrangement, perpendicular to the wire length direction, is less than 500 μm. The spacing means that wire bending processes do not need to be carried out in order to make connection to the miniature sensor.

The invention is of particular interest for guidewire sensors.

Bifilar and multifilar wires are used in guidewires for connecting the sensor at the tip of the guidewire to the back (proximal) side of the guidewire where signal processing and analysis takes place.

These extremely small wires are typically made by bonding insulated conductors in pairs, triplets or even multiple wire bundles. The gap between the conductors is determined by the insulation thickness of the individual conductors and is for example only a few micrometers.

Typical pitches of bond pads on sensors are in the range of 30 to 200 micrometers, for example 30 to 60 micrometers. This means that the conductors in a wire have to be brought to the desired pitch by a bending process. This bending process is complicated and can decrease the wire and product quality.

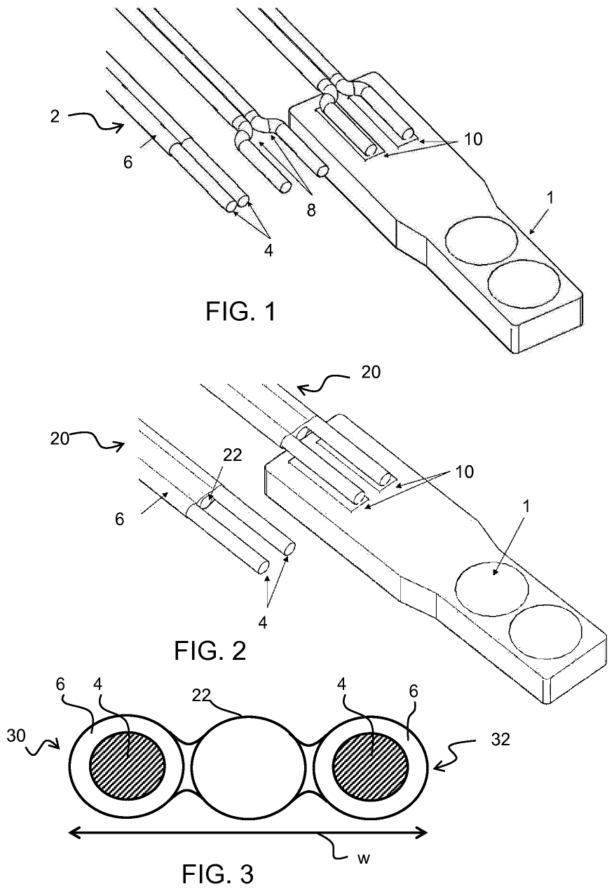

FIG. 1 shows a sensor 1 connected to a conventional conductor arrangement 2 having two conductor wires, each having a conducting core 4 and an insulating shroud 6. The conductor arrangement 2 is formed as a pair of bonded wires, connected by the insulating shroud 6 around each wire.

As shown in the left image, the insulating shroud 6 is removed from an end region to form bare wire end portions.

The bare wire portions are then bent apart at bends 8 as shown in the middle image, so that the wire spacing corresponds to a bond pad pitch.

The right image shows the bare end portions connected to bond pads 10 of the sensor module 1. This connection may be by soldering or welding.

FIG. 2 shows a similar sensor connection to a conductor arrangement 20 in accordance with the invention. The same components are given the same reference numerals as in FIG. 1.

The conductor arrangement 20 has a spacer 22 between the two conductor wires. The spacer may be formed of the same material as the insulating shroud 6 of the two wires so that they may all be bonded together. The spacer may have the shape of a dummy (i.e. non-conducting) wire between the two conductor wires. In this way, the standard process for forming a wire triplet or wire bundle may be applied to the conductor wires and spacer(s). This design avoids the need for the bending process (the middle image in FIG. 1) of forming the conductor wires with the same pitch as the pitch of the bond pads 10 on the sensor.

During the removal of the insulation 6 from the conductor wires, the spacer is also removed. This stripping process is for example performed by a laser with a wavelength which removes the organic insulation but does not attack the metal core of the conductor wires. An excimer laser with a wavelength of 248 nm is one option.

FIG. 3 shows a cross section of the conductor arrangement. There are two conductor wires 30, 32, each with a metal core 4 and an insulating shroud 6. The spacer 22 is bonded with the shrouds 6. The spacer is shown as a cylinder in this example, and it therefore functions as a dummy wire between the conductor wires. The bonding with the shrouds creates an integrated structure.

By way of example, the total width w of the conductor arrangement may be 125 μm. The core 4 of each conductor wire may have a diameter of 30 μm and a shroud thickness of 5 μm. The central spacer has the same total diameter of 40 μm as each conductor wire. This gives the example shown a pitch of 75 μm.

A cylindrical spacer design is preferred because the spacing provided is then independent of the orientation of the spacer. It also means that the collection of conductor wires and spacers may be bonded in the same way as a conventional set of conductor wires. The diameter of the central spacer cylinder is thus preferably the same as for the conductor wires, so that they may more easily be formed into a one-dimensional (flat) array before being bonded together. If more space is needed between the conductor wires a second dummy wire may for example be provided. The conductor wire diameter may be chosen so that one or more spacers of the same diameter results in the required bond pad spacing.

The individual electrical conductors are for example insulated with polyamide or polyimide by applying these materials as a liquid (in a solvent) followed by a curing step.

A bifilar structure is for example made by feeding two insulated wires and the dummy spacer wire into a process which bonds the wires for example using an epoxy. The spacer thus preferably has the form of a dummy wire so that it can be processed as if it was a conventional wire when forming the bonded overall structure. This means that standard bonding processes for an array of multifilar wires of the desired small dimensions may be used.

However, the spacer may in theory have any suitable shape for maintaining a desired spacing between the conductor wires. It may have a bar form, of a width equal to or less than the outer diameter of each wire 30, 32. In such a case, the spacer may be formed integrally with the insulating shrouds of the conductor wires as part of the process of providing an insulating shroud around the cores of the individual conductor wires. The example shown has two conductor wires. However, there may be more than two conductor wires. The multiple conductor wires are preferably formed as a flat array so that the conductor wires may be attached to a planar array of bond pads without the need to deform the conductor arrangement.

The invention may be used in any application where there is a benefit in having conductor wires having a pitch equal to the pitch of the pads of a sensor. As explained above, the invention is of particular interest for miniature sensors and conductor wires. One example of a set of dimensions has been given above.

More generally, each metal core may have a diameter of less than 100 μm for example less than 50 μm, and each insulating shroud may have a thickness of less than 40 μm for example less than 20 μm. The total width w is typically less than 500 μm.

One area of particular interest is for guidewire sensors for use as part of a guidewire of a catheter or stent delivery system.

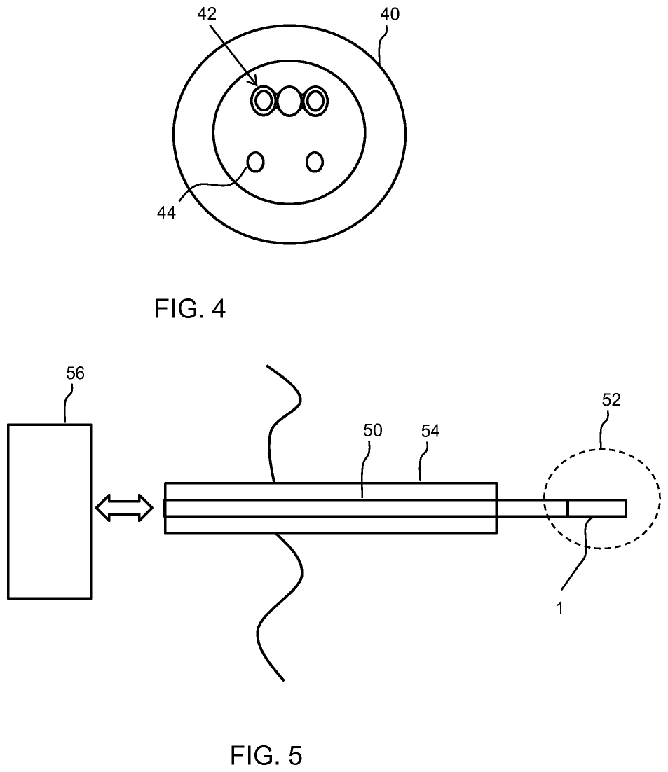

FIG. 4 shows a cross section of a guidewire, comprising an outer sheath 40 within which the electrical conductor arrangement 42 passes. There may also be optical fibers 44 for optical signals, for example from optical sensors.

FIG. 5 shows a catheter system comprising a guidewire 50 with a sensor 1 at the tip, in a region 52 of interest. A catheter 54 is guided around the guidewire 50. A signal processing system 56 receives signals from the sensor 1 which may have optical as well as electrical sensor elements. There may also be sensor elements distributed along the length of the guidewire.

A stent system may function in the same way, with the stent delivered along the guidewire.

Some examples of sensor type have been given above. There are various MEMS sensor devices which may be provided as part of a guidewire system, such as pressure sensing MEMS, flow sensing MEMS, barometer MEMS. Electrical connections may also be made in the same way to an antenna or to electronic imaging arrangements. The wire connection solution explained above may be applied to all of these possible sensors.

The example above places the sensor at the tip of the guidewire. The sensor may instead be located set back from the end of the guidewire, for example recessed into a side wall of the guide wire.

The way the guidewire is used and the details of the catheter system will not be described in detail since the conventional methods and systems may be used.

The invention may be applied to any miniaturized sensor system, for example for a sensor directly at the tip of a catheter (without needing a guidewire), or a needle or other small diameter inspection probe. There are medical as well as non-medical applications for such sensing.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A conductor arrangement for connection to a miniature sensor, comprising:
a first conductor wire;
a second conductor wire;
a dummy wire forming an insulating spacer between and bonded to the first and second conductor wires, wherein the first and second conductor wires and the dummy wire form a flat conductor array,
wherein the maximum lateral dimension (w) of the conductor arrangement, perpendicular to the wire length direction, is less than 500 μm.

2. A conductor arrangement as claimed in claim 1, wherein the first and second conductor wires each comprise a metal core and an insulating shroud.

3. A conductor arrangement as claimed in claim 2, wherein each metal core has a diameter of less than 100 μm, for example less than 50 μm.

4. A conductor arrangement as claimed in claim 2, wherein each insulating shroud has thickness of less than 40 μm, for example less than 20 μm.

5. A conductor arrangement as claimed in claim 2, wherein the spacer comprises an insulating cylinder.

6. A conductor arrangement as claimed in claim 5, wherein the spacer has an outer diameter equal to the outer diameter of each conductor wire.

7. A conductor arrangement as claimed in claim 1, wherein there are exactly two conductor wires.

8. A sensor arrangement comprising:
a sensor module, having first and second connection pads; and
a conductor arrangement as claimed in claim 1, wherein the first and second conductor wires each comprise a bare end portion which is connected to a respective one of the connection pads.

9. A sensor module as claimed in claim 8, wherein the bare end portions are straight.

10. A sensor arrangement as claimed in claim 8, wherein each bare end portion is connected to the respective connection pad by soldering or welding.

11. A sensor arrangement as claimed in claim 8, wherein the pitch of the first and second conductor wires is equal to a pitch of the first and second connection pads, wherein for example the pitch is between 30 and 200 μm.

12. A sensor arrangement as claimed in claim 8, wherein the sensor module comprises a pressure sensor and/or a flow sensor and/or a temperature sensor and/or an ultrasound transducer.

13. A sensor arrangement as claimed in claim 8, comprising a guidewire sensor arrangement.

14. A guidewire comprising a sensor arrangement as claimed in claim 8.

15. A catheter or stent system, comprising:
a guidewire as claimed in claim 14; and
a catheter or stent which is adapted to be guided over the guidewire.
